(19) United States
(12) Patent Application Publication
(10) Pub. No.: US 2004/0116496 A1
(43) Pub. Date: Jun. 17, 2004

(54) Asymmetric Disulfides and methods of using same

(76) Inventors: Lynn Kirkpatrick, Tuscon, AZ (US); Garth Powis, Tuscon, AZ (US)

Correspondence Address: Pepper Hamilton LLP, One Mellon Center, 50th Floor, 500 Grant Street, Pittsburgh, PA, 15219, US

(21) Appl. No.: 10/617,949

(22) Filed: Jul. 10, 2003

Related U.S. Application Data
(63) Continuation-in-part of application No. 10/366,751, filed on Feb. 14, 2003.
Application No. 10/366,751 is a continuation of application No. 09/132,421, filed on Aug. 11, 1998, now Pat. No. 6,552,060.
(63) Continuation-in-part of application No. 09/875,578, filed on Jun. 6, 2001, now Pat. No. 6,689,775.
Application No. 09/875,578 is a continuation of application No. 09/319,292, filed on Jun. 3, 1999, now abandoned.
Application No. 09/319,292 is a 371 of international application No. PCT/US97/22292, filed on Dec. 5, 1997.
(60) Provisional application No. 60/055,201, filed on Aug. 11, 1997.
(60) Provisional application No. 60/031,995, filed on Dec. 6, 1996.

(51) Int. Cl. A61K 9/22

(52) U.S. Cl. 514/398; 424/468

(57) ABSTRACT

The present invention is directed to a composition or formulation which includes an asymmetric disulfide which alone or in combination inhibits or interferes with cellular redox function, as well as a method of using same to restore normal cellular function. More specifically, the composition of the present invention are delivered to the patient over a period of time and interact with, interfere with, or inhibit abnormal cellular proliferation and restores or prevents inhibition of cellular apoptosis.

### RELATED APPLICATION

This application is a continuation in part of U.S. patent application Ser. No. 10/366,751 which is a continuation of U.S. Ser. No. 09/132,421 now U.S. Pat. No. 6,552,060 which claims continuing status from Provisional Patent Application Serial No. 60/055,201, filed Aug. 11, 1997 the contents each of which are incorporated herein by reference in their entirety; this application is also a continuation in part of U.S. patent application Ser. No. 09/875,578, which is a continuation of application U.S. Ser. No. 09/319,292, filed on Jun. 3, 1999, which is a national phase filing based on International Application No. PCT/US97/22292, filed on Dec. 5, 1997, which claimed the benefit of priority from U.S. Provisional Patent Application Serial No. 60/031,995, filed on Dec. 6, 1996 the contents each of which are included herein by reference in their entirety.

### GOVERNMENTAL INTERESTS

This invention was made with support from the U.S. government under a grant from the U.S. National Institutes of Health, contract number CA48725 and NIH SBIR grant No. 5R44CA07923-04. The U.S. government has certain rights in the invention.

### FIELD OF THE INVENTION

The present invention relates generally to asymmetric disulfides, and more specifically to therapeutic compositions comprised of asymmetric disulfides, said asymmetric disulfides providing a desired therapeutic activity.

### BACKGROUND OF THE INVENTION

Cellular redox systems are important to normal cellular activity. Cells maintain an intracellular environment that is reducing in the face of a highly oxidizing extra-cellular environment. Regulated alterations in the intracellular redox state (redox signaling) can modulate cellular activity, including activities such as DNA synthesis, enzyme activation, selective gene expression, cell cycle regulation, cell growth, and programmed cell death.

Abnormal cellular proliferation is one type of abnormal cell function that is a cardinal feature of human malignancy. In recent years there has been great insight into the bio-molecules that regulate cell proliferation and the pathways in which they operate. These bio-molecules have been identified as pharmacological, therapeutic, and/or diagnostic targets for agents which inhibit cellular proliferation. Abnormal cellular proliferation is most often associated with cancer and other hyperproliferative diseases.

Another type of abnormal cell function is resistance to apoptosis. Apoptosis is a form of programmed cell death characterized by membrane bleading, chromatin margination and breakdown of chromosomal DNA into nucleosome-sized fragments. Loss of apoptosis can lead to diseases such as cancer, autoimmune disease, inflammation, and hyperproliferation disease. Increased apoptosis can lead to neurodegenerative disease and destruction of tissue, as well as cardiovascular damage. Normally, when a cell sustains substantial genetic damage that cannot be repaired through normal DNA repair processes, sensory mechanisms in the cell recognize this and initiate a sequence of events which leads to the death of the cell. Apoptosis results in the death of damaged cells and protects the organism from potentially harmful genetic changes. Inhibition of apoptosis by abnormal expression of an oncogene or loss of a tumor suppressor gene can be closely associated with malignancy. As cells progress from a non-transformed state, through a pre-malignant state to a fully transformed state, the cells lose their ability to undergo apoptosis. Apoptosis is also inhibited by some viral infections.

Discovery of molecules which interfere with or inhibit cellular redox systems satisfies a need in the art by providing new diagnostic or therapeutic compositions useful in the detection, prevention, and treatment of diseases related to abnormal cellular activity (i.e., hyperproliferation or apoptosis).

Because there is a correlation between treatment of a patient with an asymmetric disulfide and white blood cell and circulating it is of great clinical importance to assure a steady level of the asymmetric disulfide for establishment of a therapeutic treatment regiment.

### SUMMARY OF THE INVENTION

The present invention pertains to asymmetric disulfides and sustained release compositions thereof and focuses on the interaction of these released disulfides with cellular signaling pathways having points of redox control. Asymmetric disulfides that inhibit or interfere with cellular redox function have strong potential applications as therapeutic agents, diagnostic tools, chemopreventative agents and chemotherapeutic agents.

The present invention also relates to methods of using asymmetric disulfides compositions for therapeutic and prophylactic treatment of a mammalian host, preferably a human. The disulfides of the present invention may be administered alone or in combination with other therapeutic agents (e.g. other anti-cancer drugs).

The present invention also relates to a composition comprised of an asymmetric disulfide and a pharmaceutically acceptable carrier of said asymmetric disulfide wherein said composition is useful in treating disease. The disulfides of the present invention may also be utilized to prevent the inhibition of apoptosis.

One aspect of the invention relates to a method of treating a patient having a cellular proliferation disease by delivering to the patient a sustained concentration of a composition that includes an asymmetric disulfide or a derivative thereof and a pharmaceutically acceptable excipient. Preferably the composition allows dosing of the patient eight times per day or less while maintaining a therapeutically acceptable concentration of the asymmetric disulfide in the patient.

An embodiment of the present invention involves a method of inhibiting tumor cell growth in a tumor that over-expresses thioredoxin comprising contacting said tumor with a cell growth inhibiting effective amount of an asymmetric disulfide.

Another embodiment of the present invention involves a method of reducing inhibition of apoptosis in tumor cells that over-express thioredoxin comprising contacting said tumor cells with an effective amount of an asymmetric disulfide.

One aspect of the invention relates to a sustained or controlled-release oral drug delivery composition for treatment of a cellular proliferation disease which includes as an active ingredient an asymmetric disulfide or a derivative thereof, which is formulated within a polymeric matrix comprising a hydrophilic, or a mixture of hydrophilic and hydrophobic polymers, and optionally having additional pharmaceutically acceptable excipients, wherein the asymmetric disulfide is released from the matrix in a sustained concentration over a time period of about 3 hours or more, and wherein the release of the asymmetric disulfide to the patient is by nearly zero-order kinetics.

Another embodiment of the invention is the use of an asymmetric disulfide in the prophylactic treatment of a patient to prevent a cellular proliferation disease. The treatment includes administering an effective amount of composition including 1-methylpropyl 2-imidazolyl disulfide to prevent abnormal cell activity in a patient.

Another embodiment of the present invention is the use of an asymmetric disulfide in combination with other chemotherapeutics to treat a cancer.

In another embodiment of the present invention the sustained release composition including the asymmetric disulfide is used to treat diseases characterized by over expression of thioredoxin in the patient such as breast cancer, renal cancer, colon cancer, and glialblastomas. The composition may also be used to treat diseases which are independent of thioredoxin expression such as FAP and angiogenisis.

This invention also relates to a method of inhibiting growth in a cell, the method being comprised of contacting the cell with an effective amount of an asymmetric disulfide. It is preferable that the asymmetric disulfide be an inhibitor of a thioredoxin/thioredoxin reductase redox system, and even more preferable that the asymmetric disulfide prevents inhibition of apoptosis. The growth in a cell is inhibited by an effective amount of disulfide, and may be additive to the known effectiveness of other active inhibitors.

Another aspect of the present invention is a method of inhibiting tumor growth in vivo comprised of administering an effective amount of an asymmetric disulfide. The method of inhibiting tumor growth involves administering the disulfide in therapeutically effective amounts as described above, and preferably includes mixing the asymmetric disulfide with pharmaceutical acceptable carrier and/or other therapeutic agent.

The present invention can also be described as being drawn to asymmetric disulfides and their formulation in compositions, as well as methods of their use to treat various diseased states. It is preferable in the asymmetrical disulfides of the present invention have respective R groups of divergent functionality. Preferably in the general formula R1-S—S-R2 one of R1 or R2 is a good leaving group and the respective other is a poor leaving group. Examples of good leaving groups are compounds which contain electron withdrawing groups or groups which delocalize the electrons of the functional groups (i.e., aromatic and imidazolyl groups). It is preferable that the aromatic groups of the present invention include heteroatoms such as oxygen, nitrogen, and sulfur. Poor leaving groups do not generally have such electron withdrawing characteristics or delocalized electrons. Thus, they do not form substantially stable species when or if they are cleared from the molecule. An example of a poor leaving group is an unsubstantiated alkane or alkyl group. The asymmetrical disulfides of the present invention are particularly useful to treat cancers, more particularly, cancers such as myeloma, cervical, lung, gastric, colon, renal, prostate, and breast cancers.

Finally, the present invention relates to a method of treating a diseased state by administering a therapeutically effective amount of an asymmetric disulfide having a predetermined IC₅₀ TR/Trx value, toxicity value, or hydrophilicity as described herein. The asymmetric disulfide preferably has IC₅₀ TR/Trx of less than 150 μg/ml, more preferably, less than 100 μg/ml, and even more preferably, less than 50 μg/ml.

### BRIEF DESCRIPTION OF THE DRAWINGS

The features, aspects and advantages of the present invention can be better understood with regard to the following description, appended claims, by referring to the accompanying drawings, where:

FIG. 1 illustrates how IV-2 treatment alters rhTRX electrophoretic mobility, experiments show that IV-2 treatment of rhTRX (lane 1) induces a downshift in rhTRX (lane 2) [IV-2/TRX] electrophoretic gel mobility. This downshift is associated with thioalkylation of conserved cystaine residues within the TRX active site. Subsequent studies demonstrated that IV-2 treatment of human carcinoma cell lines and normal, whole human blood induced the characteristic downshift in intracellular and circulating (plasma) TRX. These and subsequent studies also confirmed that chicken or human TRX recognized IV-2 modified TRX via Western blot and ELISA;

FIG. 2 illustrates that 1-hr IV-2 infusion results in a decrease in plasma TRX within first 15 min. of infusion;

FIG. 3 illustrates that 3-hr IV-2 infusion results in a sustained decease in plasma TRX within a day;

FIG. 4 illustrates that Clinical trail patients express wide range of pre-dose plasma TRX levels;

FIG. 5 illustrates that a 3-hr IV-2 infusion is more effective than 1-hr IV-2 infusion in inducting an immediate decrease in plasma TRX levels;

FIG. 6 illustrates that a 3-hr IV-2 infusion results in a significant decrease in plasma TRX at end of infusion as to Pre-Dosing levels across IV-2 treatment cohorts;

FIG. 7 illustrates that a 1-hr IV-2 infusion results in a sustained decrease in plasma TRX within cycle 1 and across IV-2 treatment cohorts in some but not all patients;

FIG. 8 illustrates that a 3-hr IV-2 infusion results in a sustained decrease in plasma TRX across IV-2 treatment cohorts;

FIG. 9 illustrates that a 3-hr IV-2 Infusion results in a sustained decrease in plasma TRX levels over a full cycle of therapy (21 day).

### DETAILED DESCRIPTION OF THE INVENTION

Before the present compositions and methods are described, it is to be understood that this invention is not limited to the particular molecules, compositions, methodologies or protocols described, as these may vary. It is also to be understood that the terminology used in the description is for the purpose of describing the particular versions or embodiments only, and is not intended to limit the scope of the present invention which will be limited only by the appended claims.

It must also be noted that as used herein and in the appended claims, the singular forms “a”, “an”, and “the” include plural reference unless the context clearly dictates otherwise. Thus, for example, reference to a “cell” is a reference to one or more cells and equivalents thereof known to those skilled in the art, and so forth. Unless defined otherwise, all technical and scientific terms used herein have the same meanings as commonly understood by one of ordinary skill in the art. Although any methods and materials similar or equivalent to those described herein can be used in the practice or testing of embodiments of the present invention, the preferred methods, devices, and materials are now described. All publications mentioned herein are incorporated by reference. Nothing herein is to be construed as an admission that the invention is not entitled to antedate such disclosure by virtue of prior invention.

The present invention relates generally to asymmetric disulfides. More specifically, the present invention relates to compounds or mixtures of compounds which include an asymmetric disulfide or biological equivalent thereof which interacts, interferes, inhibits, or competes with redox systems, particularly redox systems involving proteins having cysteine residues, and more particularly to redox systems involving thioredoxin and/or thioredoxin reductase. The asymmetric disulfides of the present invention may be used alone or in combination with other therapeutic agents or therapeutic methods. Combination therapy (i.e., chemotherapy) using two or more therapeutic drugs to treat malignant tumors in humans is specifically contemplated herein. For cancer, therapeutic or anti-cancer drugs may include anti-metabolites, alkylating agents, antibiotics, tubulant binders, etc. Combinations of drugs are administered in an attempt to obtain a synergistic cytotoxic effect on most cancers, e.g., carcinomas, melanomas, lymphomas and sarcomas, and to reduce or eliminate emergence of drug-resistant cells and to reduce side effects to each drug.

As used herein, the term asymmetric disulfide means any compound having a sulfur-sulfur linkage which is not a mirror image of itself when split down the sulfur-sulfur bond. When speaking of a particular asymmetric disulfide, the term includes all biochemical equivalents (i.e. salts, precursors, and basic form) of the particular asymmetric disulfide being referenced (i.e., reference to n-butyl imidazolyl disulfide includes the salt thereof). This term specifically includes disulfides having the general formula of R₁-S—S-R₂ as well as (bis)disulfides having the general formula of R₁-S—S-Y—S—S—R₂ wherein R₁, R₂, and Y may be any chemical substituent, but is preferably selected from the group consisting of imidazoles, thiadiazolyls, thiazolyls, benzimidazolyls, purinyls, phenyl, benzyl, phenylethyl, pyridine, pyrimidine, benzoxazole, benzthiazolyls, alkyl, cycloalkyl, hydroxylalkyl, carboxyalkyl, haloalkyl, and cycloalkanone.

When the term asymmetric disulfide is used it means that the groups on either side of a disulfide linkage are not the same. In the case of disulfides having the formula R—S—S—R this asymmetric relation may be represented by R₁—S—S—R₂. In the case of (bis) disulfide compounds although R₁ and R₂ may not be different, and the overall compound may be “symmetrical” around the center of the formula, that is, in the formula R₁—SS—Y—S—S—R₂, R₁ and R₂ may be the same group, the term asymmetrical as used herein refers to the fact that when either sulfur-sulfur linkage is split down the middle, the disulfides are asymmetrical (i.e. R—S—S—Y—S—}) are not equivalent. By this definition and as used herein all (bis) disulfide compositions are asymmetrical.

The preferred asymmetric disulfides of the present invention include imidazolyl disulfide, thiadiazolyl disulfide, mercaptothiadiazolyl disulfide, thiazolyl disulfide, phenyl disulfide, benzyl disulfide, phenylethyl disulfide, nicotinic acid disulfide, pyrimidine disulfide, benzoxazolyl disulfide, benzothiazolyi disulfide, benzimidazolyl disulfide, purinyl disulfide, cycloalkyl disulfide, captopril disulfide, and menthone disulfide.

As used herein, the term “prophylactic or therapeutic” treatment refers to the administration to the host or subject of asymmetric disulfides before or after onset of the biological damage. If the asymmetric disulfides and/or biological agent(s) are administered prior to exposure to the agent causing the biological damage or to prevent occurrence of the disease, the treatment is prophylactic (i.e., it protects the host against the damage), whereas if it is administered after exposure to the agent causing the damage, the treatment is therapeutic (i.e., it alleviates the existing disease or damage).

As used herein “to mix”, “mixing”, or “mixture(s)” means any mixing of an asymmetric disulfide with another agent or a pharmaceutically acceptable carrier of said asymmetric disulfide: i) prior to administration (“in vivo”); 2) by simultaneous and/or consecutive but separate intravenous lines of disulfide and other agent or carrier to cause “in vivo mixing”; and 3) the administration of one or the other of disulfide and agent or carrier consecutively, preferably within 48 hours of one or the other (“delayed in vivo mixing” or “saturation”).

As used herein, the term “about” means plus or minus 10% of the number to which reference is being made. For example, about 10 grams means in the range of 9-11 grams.

As used herein, IC₅₀ refers to the concentration causing 50% inhibition in activity in the system being measured. For example, in the thioredoxin reductase/Trx insulin reduction assay, IC₅₀ is defined as that concentration of inhibitor which causes a 50% decrease in the reduction of insulin by thioredoxin reductase/thioredoxin. When referring to the particular system being analyzed IC₅₀ is typically followed by an abbreviation referring to that system (i.e., IC₅₀TR/Trx for the above described thioredoxin redox system which is comprised of thioredoxin reductase and thioredoxin).

As used herein, GI₅₀ refers to that concentration of inhibitor which produces a mean 50% growth inhibition. Similar to IC₅₀, GI₅₀ normally designates the system being analyzed or the type of cell lines being tested. For example GI₅₀ (all tumors) as used herein refers to the mean growth inhibition in all 60 cell lines of the National Cancer Institute, while GI₅₀ (leukemias) refers to a mean 50% growth inhibitor for leukemia cell lines of the National Cancer Institute.

As used herein, the term “leaving group” refers to a stable species that can be detached from a molecule during a reaction and “good leaving group” refers to those species that can be displaced by a nucleophillic attack on a sulfur of an asymmetric disulfide or a (bis) disulfide. Preferably, the good leaving group includes an electron withdrawing group such as a carbonyl or a group which provides for electron delocalization such as an aromatic group.

The terms “patient” and “subject” mean all mammalian animals including humans. Examples of patients or subjects include but are not limited to humans, cows, dogs, cats, goats, sheep, and pigs.

The pharmaceutical compositions utilized in this invention may be administered by any number of routes, including but not limited to, oral, intravenous, intramuscular, intraarterial, intramedullary, intrathecal, intraventricular, transdermal, subcutaneous, intraperitoneal, intranasal, enteral, topical, sublingual, intracisternally, intra-vaginally, intravesically, locally (as powders, ointments, or drops), or rectally. In addition to the active ingredients, the compositions may contain suitable pharmaceutically-acceptable carriers comprising excipients and auxiliaries which facilitate processing of the active compounds into preparations which can be used pharmaceutically. As used herein, the term “pharmaceutically acceptable carrier” refers to a carrier medium which does not interfere with the effectiveness of the biological activity of the active ingredients and which is not toxic to the hosts to which it is administered. The compounds of the present invention can be administered to a patient either alone, as part of a pharmaceutical composition, or with other chemotherapeutic compounds or radiological treatments as part of a chemotherapeutic regiment.

As used herein, the term “pharmacologically effective amounts” refers to the amount of the referenced component which results in an increase survival of the host, or results in a desirable clinical outcome. The “therapeutic index” is defined for purposes herein in terms of efficacy (e.g., extent of tumor or infection reduction or other cure) and/or in terms of toxicity to the host. For non-human hosts, if the efficacy increases at least 50% over the efficacy using an excipient control (e.g., phosphate buffered saline) and the ratio of mean body weight at the end of the evaluation period for efficacy response to mean body weight at the start of treatment is at least 0.90 (i.e., no greater than 10% body weight loss), the therapeutic index has increased. The ratio of mean body weights indicates the extent of toxicity, with a value of 1 indicating no toxicity. For non-human hosts being treated for cancer, the extent of efficacy achieved may be measured by the ratio of mean tumor volume at the end of the evaluation period to mean tumor volume at the start of treatment. A reduction in the ratio of at least 50% of treated over excipient control indicates increased efficacy. The most preferred doses, schedules, and types of therapeutic agents are those that achieve a mean tumor volume ratio of between 0 and 5, with a value of 0 being optimum and indicating a cure. For human hosts, if the efficacy increases at least 50% upon treatment with the therapeutic agents and the toxicity is acceptable (i.e., no more than fever, chills, and/or general malaise) the therapeutic index has increased. For human hosts being treated for cancer, the extent of efficacy is generally ascertained in the clinic by measuring the perpendicular diameters of the products of all measured disease. The effect of the doses may diminish with time. For humans the dose may be repeated for months or even years.

A “therapeutically effective dose” refers to that amount of active ingredient, for example, asymmetric disulfide compound, which ameliorates the symptoms or condition. A therapeutically effective amount is an amount of an asymmetric disulfide compound that when administered to a patient or subject, ameliorates a symptom of the cellular proliferation disease or establishes normal cellular apoptosis in the patient. Therapeutic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The therapeutic index can be defined as the dose ratio between therapeutic and toxic effects (the ratio LC₅₀/ED₅₀). Pharmaceutical compositions which exhibit large therapeutic indices are preferred.

The present invention provides a method to interfere with or inhibit abnormal cellular proliferation and restores or prevents inhibition of cellular apoptosis by administering to a patient or subject having a such diseases a therapeutically amount of an asymmetric disulfide, analogs, pharmaceutically acceptable salts, and prodrugs thereof.

The method of the present invention involves administering to a mammalian host, preferably a human host, pharmacologically effective amounts of an asymmetric disulfide. The asymmetric disulfides may be combined in vitro before administration or separately administered to the host with other anti-cancer agents, chemotherapeutics or radiological treatments, either concurrently or simultaneously, with administration generally taking place up to 24 hours before or after the administration of the other biological active agents.

The dose and dosage regimen of the asymmetric disulfide to the patient will depend mainly on whether the inhibitors are being administered for therapeutic or prophylactic purposes, separately or as a mixture, the type of biological damage and host, the history of the host, and the type of inhibitors or biologically active agent. The amount must be effective to achieve an enhanced therapeutic index as defined herein. It is noted that humans are treated longer than the mice and rats with a length proportional to the length of the disease process and drug effectiveness. The doses may be single doses or multiple doses over a period of several days, but single doses are preferred. The specific dosage used, however, can vary. For example, the dosage can depend on a numbers of factors including the requirements of the patient, the severity of the condition being treated, and the pharmacological activity of the compound being used. The determination of optimum dosages for a particular patient is well known to those skilled in the art.

A therapeutic purpose is achieved as defined herein is when the treated hosts exhibit improvement against disease or infection, including but not limited to improved survival rate, more rapid recovery, or improvement or elimination of symptoms. If multiple doses are employed, as preferred, the frequency of administration will depend, for example, on the type of host and type of cancer, dosage amounts, etc. For some types of cancers or cancer lines, four times daily administration or less may be effective, daily administration may be effective, whereas for others, administration every other day or every third day may be effective, but daily administration ineffective. The practitioner will be able to ascertain upon routine experimentation which route of administration and frequency of administration are most effective in any particular case. The dosage amounts for cancer which appear to be most effective herein are those that result in regression in size of the tumor or complete disappearance or non-reappearance of the tumor, and are not toxic or are acceptably toxic to the host patient. The dosage amounts for establishing cellular apoptosis which appear to be most effective herein are those that result in normal cellular apoptosis for the patient, and are not toxic or are acceptably toxic to the host patient. The optimum dose levels may also depend on sequence of administration, existing tumor burden, are the type of precursor.

Compounds and agents of the present invention, in conjunction with a pharmaceutically acceptable carrier, may be used for any of the therapeutic effects, discussed above. Such compositions may be in the form of an agent in combination with at least one other agent, such as stabilizing compound, which may be administered in any sterile, bio-compatible pharmaceutical carrier, including, but not limited to, saline, buffered saline, dextrose, and water. The compositions may be administered to a patient alone, or in combination with other agents, drugs or hormones. Pharmaceutically-acceptable carriers may also be comprised of excipients and auxiliaries which facilitate processing of the active compounds into preparations which can be used pharmaceutically. Further details on techniques for formulation and administration may be found in the latest edition of Remington's Pharmaceutical Sciences (Maack Publishing Co., Easton, Pa.) hereby incorporated herein by reference in its entirety.

The pharmaceutical composition may be provided as a salt and can be formed with many acids, including but not limited to, hydrochloric, sulfuric, acetic, lactic, tartaric, malic, succinic, etc. Salts tend to be more soluble in aqueous or other protonic solvents than are the corresponding free base forms. In other cases, the preferred preparation may be a lyophilized powder which may contain any or all of the following: 1-50 mM histidine, 0.1%-2% sucrose, and 2-7% mannitol, at a pH range of 4.5 to 5.5, that is combined with buffer prior to use.

For topical or nasal administration, penetrants appropriate to the particular barrier to be permeated are used in the formulation. Such penetrants are generally known in the art.

The pharmaceutical compositions of the present invention may be manufactured in a manner that is known in the art, e.g., by means of conventional mixing, dissolving, granulating, dragee-making, levigating, emulsifying, encapsulating, entrapping, or lyophilizing processes.

After pharmaceutical compositions have been prepared, they can be placed in an appropriate container and labeled for treatment of an indicated condition, For administration of thioredoxin reductase/Trx inhibitors, such labeling would include amount, frequency, and method of administration.

Those skilled in the art are easily able to identify patients or subjects having a cellular proliferation disease or a condition characterized by inhibition of cellular apoptosis. Examples of such diseased patients include those who are suffering from cancers such as breast, renal, and colon, glialblastomas, or FAP polyps.

For any of the asymmetric disulfide compounds discussed herein, the therapeutically effective dose can be estimated initially either in cell culture assays, e.g., of neoplastic cells, or in animal models, usually mice, rabbits, dogs, or pigs. The animal model may also be used to determine the appropriate concentration range and route of administration. Such information can then be used to determine useful doses and routes for administration in humans.

The exact dosage will be determined by the practitioner, in light of factors related to the subject that requires treatment. Dosage and administration are adjusted to provide sufficient levels of the active moiety or to maintain the desired effect, Factors which may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

The asymmetric disulfides preferably inhibit or interfere with thioredoxin redox system, and more preferably, the disulfides inhibit thioredoxin reductase or thioredoxin. The asymmetric disulfide and pharmaceutically acceptable carrier are preferably formulated or administered in a therapeutically effective amount. The therapeutically effective amount is preferably in a range from about 0.05 mg/kg/day to about 5,000 mg/kg/day, more preferably in a range from about 0.5 mg/kg/day to about 500 mg/kg/day, more preferably in a range of about 1 mg/kg/day to about 50 mg/kg/day, and more preferably yet, the therapeutically effective amount is in a range from about 2 mg/kg/day to about 20 mg/kg/day, and most preferably the therapeutically effective amount is in a range from about 5 mg/kg/day to about 10 mg/kg/day.

Pharmaceutical formulations suitable for parenteral administration may be formulated in aqueous solutions, preferably in physiologically compatible buffers such as Hanks's solution, Ringer's solution, or physiologically buffered saline. Aqueous injection suspensions may contain substances which increase the viscosity of the suspension, such as sodium carboxyrmethyl cellulose, sorbitol, or dextran. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Optionally, the suspension may also contain suitable stabilizers or agents which increase the solubility of the compounds to allow for the preparation of highly concentrated solutions.

Compositions suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents or vehicles include water, ethanol, polyols (propyleneglycol, polyethyleneglycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions and by the use of surfactants.

Compositions suitable for parenteral injection may also contain adjuvants such as preserving, wetting, emulsifying, and dispensing agents. Prevention of the action of microorganisms can be ensured by various antibacterial and antifungal agents, for example, parabens, chlorobutanol, phenol, sorbic acid, and the like. It may also be desirable to include isotonic agents, for example, sugars, sodium chloride, and the like. Prolonged absorption of the injectable pharmaceutical form can be brought about by the use of agents delaying absorption, for example, aluminum monostearate and gelatin.

The formulation for percutaneous administration is preferably formed by adding a given amount of the asymmetric disulfide to a solution of a solidifying or gel-forming agent or a mixture thereof in a suitable solvent or mixture of solvents and mixing or heating the mixture thereby obtained so as to form said solid, semi-solid or mucilaginous medium. The solvent used is preferably water. However, the solvent used may also suitably be an alcohol such as ethanol or stearyl alcohol, glycerol, propylene glycol, polyethlene glycol or a silicone or a mixture thereof, including a mixture with water. The formulation for percutaneous administration according to the invention may also include one or more auxiliary agent(s) selected from an antimicrobial agent, a preservative, an antioxidant, a pH-controlling agent, a plasticizer, a surfactant, a penetration enhancer, a humectant, a local anesthetic, an anti-irritant agent or a rubefacient or a mixture thereof.

The formulation for percutaneous administration when in the form of a solid or semi-solid preferably has a surface area in the range 2 to 15 cm², more especially 5 to 10 cm₂. The thickness of the formulation for percutaneous administration is preferably in the range 0.5 to 3 mm, more especially in the range 1 to 2 mm.

Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient.

Pharmaceutical preparations for oral use can be obtained through combination of active compounds with solid excipient, optionally grinding a resulting mixture, and processing the mixture of granules, after adding suitable auxiliaries, if desired, to obtain tablets or dragee cores. Suitable excipients are carbohydrate or protein fillers, such as sugars, including lactose, sucrose, mannitol, or sorbitol; starch from corn, wheat, rice, potato, or other plants; cellulose, such as methyl cellulose, hydroxypropylmethyl-cellulose, or sodium carboxymethylcellulose; gums including arabic and tragacanth; and proteins such as gelatin and collagen. If desired, disintegrating or solubilizing agents may be added, such as the cross-linked polyvinyl pyrrolidone, agar, alginic acid, or a salt thereof, such as sodium alginate.

Dragee cores may be used in conjunction with suitable coatings, such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound. i.e., dosage.

Pharmaceutical preparations which can be used orally include push-fit capsules made of gelatin, as well as soft, sealed capsules made of gelatin and a coating, such as glycerol or sorbitol. Push-fit capsules can contain active ingredients mixed with a filler or binders, such as lactose or starches, lubricants, such as talc or magnesium stearate, and, optionally, stabilizers. In soft capsules, the active compounds may be dissolved or suspended in suitable liquids, such as fatty oils, liquid, or liquid polyethylene glycol with or without stabilizers.

A “sustained release” composition refers to a composition, such as a tablet or IV, which is capable of releasing an asymmetric disulfide to the body for a prolonged period of time, e.g., for at least about 3 hours, preferably for at least about 8 hours, more preferably for at least about 12 hours, and most preferably for up to about 18-24 hours. Preferably, a sustained release tablet releases the asymmetric disulfide from the tablet gradually into the body and maintains a therapeutically effective amount of the asymmetric disulfide in the patient with three times per day dosing or less and more preferably twice daily dosing or less. For example, a sustained release tablet that is designed to release an active agent for about 18-24 hours preferably has the following dissolution specification: no more than 40% of the active agent (e.g., by weight) released in 1 hour, about 70-85% of the active agent released in 12 hours, and no less than about 80% of the active agent released at 24 hours. In another example, a sustained release tablet is designed to release the active agent at a nearly linear zero order rate (typically when the active agent dissolution is measured up to 70% of the active agent release).

While the drug/matrix may be formulated into granules, capsules or other solid pharmaceutical compositions, the erodible tablet form is most preferred. Solid dosage forms for oral administration include capsules, tablets, pills, powders, and granules. In such solid dosage forms, the active compound is admixed with at least one inert customary excipient (or carrier) such as sodium citrate or dicalcium phosphate or (a) fillers or extenders, as for example, starches, lactose, sucrose, glucose, mannitol, and silicic acid, (b) binders, as for example, carboxymethylcellulose, alignates, gelatin, polyvinylpyrrolidone, sucrose, and acacia, (c) humectants, as for example, glycerol, (d) disintegrating agents, as for example, agar-agar, calcium carbonate, potato or tapioca starch, alginic acid, certain complex silicates, and sodium carbonate, (e) solution retarders, as for example, paraffin, (f) absorption accelerators, as for example, quaternary ammonium compounds, (g) wetting agents, as for example, cetyl alcohol, and glycerol monostearate, (h) adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents.

Solid compositions of a similar type may also be employed as fillers in soft and hard-filled gelatin capsules using such excipients as lactose or milk sugar as well as high molecular weight polyethyleneglycols, and the like.

Solid dosage forms such as tablets, dragees, capsules, pills, and granules can be prepared with coatings and shells, such as enteric coatings and others well known in the art. They may contain opacifying agents, and can also be of such composition that they release the active compound or compounds in a certain part of the intestinal tract in a delayed manner. Examples of embedding compositions which can be used are polymeric substances and waxes. The active compounds can also be in micro-encapsulated form, if appropriate, with one or more of the above-mentioned excipients.

A polymer matrix/asymmetric disulfide formulation is preferably fabricated into tablets, capsules or granules for oral use. Rate of asymmetric disulfide release from the tablets is controlled by the erosion mechanism of the polymer from which the asymmetric disulfide is released within the patient by approximately zero-order kinetics. Preferably the formulation maintains a therapeutically effective concentration of the asymrnetric disulfide in the patient with a dosage of four times daily or less. Examples of hydrophilic polymers which are suitable as the matrix for the approximately zero-order release kinetics of the asymmetric disulfide of the invention are hydrophilic cellulose derivatives.

Liquid dosage forms for oral administration include pharmaceutically acceptable emulsions, solutions, suspensions, syrups, and elixirs. In addition to the active compounds, the liquid dosage forms may contain inert diluents commonly used in the art, such as water or other solvents, solubilizing agents and emulsifiers, as for example, ethyl alcohol, isopropyl alcohol, ethyl carbonate, ethyl acetate, benzyl alcohol, benzyl alcohol, benzyl benzoate, propyleneglycol, 1,3-butyleneglycol, dimethylformamide, oils, in particular, cottonseed oil, groundnut oil, corn germ oil, olive oil, castor oil and sesame oil, glycerol, tetrahydrofurfuryl alcohol, polyethyleneglycols and fatty acid esters of sorbitan or mixtures of these substances, and the like.

Besides such inert diluents, the composition can also include adjuvants, such as wetting agents, emulsifying and suspending agents, sweetening, flavoring, and perfuming agents.

Suspensions, in addition to the active compounds, may contain suspending agents, as for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like.

Compositions for rectal administrations are preferably suppositories which can be prepared by mixing the compounds of the present invention with suitable non-irritating excipients or carriers such as cocoa butter, polyethyleneglycol or a suppository wax, which are solid at ordinary temperatures but liquid at body temperature and therefore, melt in the rectum or vaginal cavity and release the active component.

Dosage forms for topical administration of a compound of this invention include ointments, powders, sprays, and inhalants. The active component is admixed under sterile conditions with a physiologically acceptable carrier and any preservatives, buffers, or propellants as may be required. Ophthalmic formulations, eye ointments, powders, and solution are also contemplated as being within the scope of this invention.

The term “pharmaceutically acceptable salts, and prodrugs” as used herein refers to those salts, and prodrugs of the asymmetric disulfides of the present invention which are, within the scope of sound medical judgment, suitable for use in contact with the tissues of patients without undue toxicity, irritation, allergic response, and the like, commensurate with a reasonable benefit/risk ratio, and effective for their intended use, as well as the zwitterionic forms, where possible, of the compounds of the invention. The term “salts” refers to the relatively non-toxic, inorganic and organic acid addition salts of compounds of the present invention. These salts can be prepared in situ during the final isolation and purification of the compounds or by separately reacting the purified compound in its free base form with a suitable organic or inorganic acid and isolating the salt thus formed. Representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactobionate and laurylsulphonate salts, and the like. These may include cations based on the alkali and alkaline earth metals, such as sodium, lithium, potassium, calcium, magnesium, and the like, as well as non-toxic ammonium, quaternary ammonium and amine cations including, but not limited to ammonium, tetramethylammonium, tetraethylammonium, methylamine, dimethylamine, trimethylamine, triethylamine, ethylamine, and the like. (See, for example, S. M. Barge et al., “Pharmaceutical Salts,” J. Pharm. Sci., 1977, 66:1-19 which is incorporated herein by reference.).

The term “prodrug” refers to compounds that are rapidly transformed in vivo to yield the parent compounds of the above formula, for example, by hydrolysis in blood. A thorough discussion is provided in T. Higuchi and V. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987, both of which are incorporated herein by reference.

In addition, the compounds of the present invention can exist in unsolvated as well as solvated forms with pharmaceutically acceptable solvents such as water, ethanol, and the like. In general, the solvated forms are considered equivalent to the unsolvated forms for the purposes of the present invention.

In addition, it is intended that the present invention cover compounds made either using standard organic synthetic techniques, including combinatorial chemistry or by biological methods, such as through metabolism.

One component of the sustained release delivery system may be a polymeric matrix comprising at least one hydrophilic polymer but may contain two or more hydrophilic polymers in admixture as described in U.S. Pat. No. 6,296,873 and U.S. Pat. No. 6,419,945, the contents of which are incorporated herein by reference in their entirety. When hydrated, the polymer forms a gel layer around the dry tablet core. The matrix of the invention is made of low or high viscosity erodible polymers or mixtures thereof.

Suitable polymers are mixed with drug in a weight ratio of polymer to drug from about 1:99% to about 99:1%, preferably from about 5:95% to about 90:10%, most preferably from about 10:90% to about 80:20%, depending on the viscosity grade of the polymer, on the tablet dimension and shape and on the desired release rate.

Preferred hydrophilic cellulose derivatives are methyl cellulose, hydroxypropyl methylcellulose, hydroxyethyl cellulose, hydroxypropyl cellulose, hydroxyethyl methylcellulose, carboxy methylcellulose and sodium carboxy methylcellulose. The most preferred hydrophilic cellulose derivative is hydroxypropyl methylcellulose (HPMC).

HPMC is particularly preferred for use with an asymmetric disulfide or an asymmetric disulfide derivative because of very low water solubility of asymmetric disulfide or its derivative. HPMC is available in a low, normal or high viscosity grades. The viscosity of the polymer controls the release rate of the drug from the formulation and affects its release kinetics. Specific HPMCs which are most suitable for the current formulation are Methocel K100M, K15M, F4M, E4M, K4M, K100LV, K3, E15LV, E15LN, E15CLV, E5O, E5 and E3, commercially available from Colorcon, Orpington, England.

The polymers useful in the current invention are preferably methocels having viscosity grade from about 3 to about 100,000 mPa sec at 2% of concentration at 20. degree. C.

The hydrophilic polymers are preferred because they allow an approximately zero-order release rate of the active asymmetric disulfide from the polymeric matrix formulation, such as an erodible tablet. When the tablet erodes within the digestive system, the asymmetric disulfide or its derivatives are released from the matrix with an approximately zero-order release kinetics and are readily absorbed.

In general, the ratio of drug to polymer is varied depending on the size and shape of the tablet, the desired asymmetric disulfide release rate, and on the molecular weight and viscosity grade of the polymer, which in general will be from about 3 to about 100,000 mPa s, preferably from about 5 to about 15,000 mPa s at 2% concentration at 20° C. temperature.

Typically, when the viscosity grade of the polymer is higher, the release of the drug is slower. When the shape of the tablet is flatter, i.e. when the ratio of a tablet diameter to a tablet width is higher, the drug release is faster. Taking these parameters into consideration, the tablet is formulated according to the drug release requirement to be more flat, that is, having a large surface for faster release or more cylindrical, that is, having smaller surface for slower release. Additionally, for slower release of the drug the higher viscosity grade polymer is used and vice-versa.

The polymeric matrix of the drug delivery of the invention may additionally also contain a hydrophobic polymer. Suitable hydrophobic polymers are hydrophobic cellulose derivatives, such as ethyl cellulose, fats, such as glycerol palmitostearate, waxes, such as beeswax, glycowax, castrowax, carnaubawax, glycerol monostearate or stearylalcohol, hydrophobic polyacrlamide derivatives and hydrophobic methacrylic acid derivatives.

When using a hydrophobic polymer, in order to provide approximately zero-order release rate of the asymmetric disulfide, such hydrophobic polymer is used only in a mixture of hydrophilic and hydrophobic polymers. In such a mixture, the hydrophobic polymer controls the water penetration rate into the delivery system. Incorporation of hydrophobic polymer into the polymer matrix and the ratio of hydrophilic to hydrophobic polymer thus changes the erosion characteristics of the tablet. The hydrophobic polymer shows down the water penetration into the tablet and thus slows the tablet erosion.

The hydrophobic polymer is added to the hydrophilic polymer in amount from about 0.1 to about 10%, preferably from about 1% to about 5%, of the total polymer. Ratios of hydrophilic to hydrophobic polymer are from about 99.9:0.1 to about 90:10, preferably from about 99:1 to about 95:5.

As used herein, the term “biological damage” refers to any damage to cellular components, body tissue or other body parts or functions sustained by the host, as a result of abnormal redox in the host (i.e. abnormal cellular proliferation).

The term “cancer” as used herein refers to any neoplastic disorder, including such cellular disorders for example, renal cell cancer, Kaposi's sarcoma, chronic leukemia, breast cancer, sarcoma, ovarian carcinoma, rectal cancer, prostate cancer, throat cancer, melanoma, colon cancer, bladder cancer, mastocytoma, lung cancer and gastrointestinal or stomach cancer. Preferably, the cancer is colon cancer, breast cancer and gasteric cancer, melanoma, renal cell cancer, sarcoma, lung cancer, adenocarcinoma, prostate or breast cancer. Even more preferably colon, breast, lung, gastric and prostate cancer.

The thioredoxin redox couple (TR/Trx) is a ubiquitous redox system found in both prokaryotic and eukaryotic cells. The thioredoxin system is comprised primarily of two elements: thioredoxin and thioredoxin reductase. Thioredoxin reductase is a NADPH-dependent selenium containing flavoprotein that catalyzes the reduction of thioredoxin. E. coli thioredoxin reductase is a 70 kDa homodimer. The active site cysteine residues, Cys-135 and Cys-138, receive electrons from FADH₂ and transfer them to the active cysteine bond of thioredoxin. During reduction, thioredoxin reductase undergoes a conformation change which protects the reduced active site cysteines from the aqueous phase, preventing spontaneous oxidation. Upon binding of oxidized thioredoxin to the active site, thioredoxin reductase undergoes a conformation change to expose the active site cysteines, allowing reduction of thioredoxin's cystine bond.

Human recombinant thioredoxin has been shown to stimulate the proliferation of human epithelial cancer cells. This appears to be due to thioredoxin's ability to enhance the activity of endogenously produced growth factors, either by acting on the factors themselves, or by affecting the factors' interaction with its cell surface receptor. For example, thioredoxin at nanomolar levels produces a 10³ fold enhancement of the growth stimulating activity of interleukin-2 and a 10² fold enhancement of the activity of basic-fibroblast growth factor with MCF-7 human breast cancer cells. Mutant redox-inactive forms of thioredoxin lacking the active site cysteine residues and E. coli thioredoxin are devoid of growth stimulating activity. It has been found that exogenously added thioredoxin stimulates mouse fibroblasts and a number of human solid tumor cells lines. Thioredoxin stimulates cell growth up to 90% as effectively as 10% fetal bovine serum stimulation. This is a characteristic exhibited by few other growth factors. One exception to this appears to be HepG2 cells whose proliferation is stimulated by thioredoxin in serum free medium, but is inhibited in the presence of 0.5% serum.

In addition to its involvement in cellular proliferation, the TR/Trx system also appears to be involved or associated with apoptosis. Apoptosis has been associated with normal cellular behavior. There is now considerable evidence that an increase in reactive oxygen species constitutes an intracellular signal that can lead to apoptosis. Apoptosis can be induced in a number of cell systems by H₂O₂, reactive oxygen species generated by the redox cycling of quinones and radiation. It appears that c-myc, which is essential for apoptosis in many systems, is induced by H₂O₂ and reactive oxygen species. Hypoxia and antioxidants inhibit apoptosis induced by these treatments. Thioredoxin protects lymphoma cells against TNF-α-mediated cell killing. The survival of embryonic mouse neurons is enhanced by thioredoxin, as well as by 2-mercaptoethanol and N-acetylcysteine. In the same studies, U251 astrocytoma cells were seen to produce increased levels of thioredoxin in response to H₂O₂ treatment. Elevated thioredoxin levels have also been observed in glial cells of the gerbil brain during reperfusion after ischaemia. Thus, thioredoxin secreted by glial cells may protect neurons, in vivo, from oxidative stress-induced cell death.

PX-12 has been found to be well tolerated and to have a wide safety window. There have been no dose limiting toxicities between 9 and 226 mg/m². PX-12 is rapidly metabolized and the metabolite is expelled through the lungs, producing a cough (Grade 1-2) on the first day of each cycle in all cohorts receiving 18 mg/m² per hour or higher. The cough appears within 3-5 minutes after the start of the infusion and lasts intermittently for 10-15 minutes. Cough has been found to be minimal or absent on days 2-5 of treatment.

There is a need for drugs and method of treatment of patients which will inhibit tumor cell growth and prevent aggressive cancer disease. The asymmetric disulfides of the present invention provide the ability to alter cellular redox in such a fashion as to manipulate the growth regulating proteins associated with undesirable health conditions. Potential therapy of pancreatic cancer is an example of the type of application for which the present invention may be utilized.

Pancreatic adenocarcinoma is a devastating disease which continues to pose a major challenge to oncologists. It is estimated that 28,300 new cases of pancreatic cancer will be diagnosed in the United States each year, making it the third most common malignancy of the digestive tract. However, due to the highly lethal nature of this disease, the mortality rate virtually parallels the incidence rate, and pancreatic cancer is the fourth most common cause of cancer-related death. Early diagnosis of pancreatic cancer is usually delayed because the initial clinical signs and symptoms are vague and non-specific. By the time the diagnosis is made, the cancer often is locally advanced or metastatic (usually to regional lymph nodes and liver), and is seldom amenable to complete surgical resection The most common presenting symptoms include weight loss, epigastric and/or back pain, and jaundice unresectable at the time of diagnosis. Gemcitabine, a deoxycytidine analogue, has been approved in the United States for first line treatment of patients with pancreatic cancer. Studies have shown that gemcitabine offers a small survival advantage over 5-fluorouracil and an increased level of palliation as evidenced by clinical endpoints of pain intensity, analgesic use, and performance status. While these results are encouraging, the overall five-year survival rate of patients across all stages remains at 4%, with the majority of patients dying within one year. Clearly, more effective treatments for pancreatic cancer are desperately needed.

It is proposed that attacking the pancreatic cancer cells Trx-1/HIF-1 hypoxia survival pathway is a novel and selective way of treating pancreatic cancer, where this rapidly growing tumor frequently outstrip its blood supply and becomes hypoxic at the growing edge of the tumor. In pancreatic cancer the hypoxia is exacerbated by the fact that the large vasculature is poorly developed. A recent study by Koong et al of intraoperative measurements of oxygen levels employing a quantitative computerized polarographic needle electrode technique has shown high levels of hypoxia and regions of extremely low oxygenation in the pancreatic tumor, with the adjacent normal pancreas having normal oxygenation. Hypoxia presents a very hostile environment for cells and cancer cells to adapt to hypoxia through the constitutive elevation of HIF-1. Genes induced by HIG-1 allow the cancer cells to survive hypoxia by changing to a glycolytic metabolism, they become resistant to programmed cell death (apoptosis) and undergo migration to potentially less hypoxic areas of the body (metastasis). Even more important, hypoxic cancer cells produce factors such as vascaular endothelial growth factor (VEGF) and FGF-2 that stimulate new blood vessel formation from existing vasculature (angiogenesis) leading to increased tumor oxygenation and growth. Angiogenesis is absolutely essential for solid tumor growth and metastasis and hypoxic tumors are highly angiogenic. Pancreatic cancers have high levels of angiogenic factor expression and it has been shown that angiogenesis correlates with poor prognosis for patients with pancreatic cancer. Paradoxically, hypoxia also causes an increase in the formation of damaging reactive oxygen species bymitochondrial complex III. Cancer cells adapt to the increase in ROS by an increase in antioxidant mechanisms, particularly those medicated by Trx family members. However, the price paid for increased oxidant protection by Trx-1 is an increase in tumor growth due to more rapid growth, decreased apoptosis and increased angiogenesis resulting in decreased patient survival.

Hence, since we have demonstrated that PX-12 not only reduces Trx-1 levels in cancer patients, but it also reduces HIF-1\acute{α} and VEGF levels in human tumor xenografts in animal models, we propose to evaluate PX-12 as a new therapy for treating pancreatic cancer, a cancer which not only has high levels of the target, Trx-1, but also is hypoxic with elevated HIF-1\acute{60}.

A composition for such treatment is the sustained release delivery of an asymmetric disulfide composition. The composition includes an asymmetric disulfide or the derivative thereof, preferably 1-methylpropyl 2-imidazolyl disulfide, and a matrix such as hydrophilic polymer or gelatin. Preferably the composition erodes and releases the asymmetric disulfide or the derivative thereof in a patient and maintains in the patient a therapeutically effective concentration of the asymmetric disulfide in the patient for at least three hours. The composition may also include a pharmaceutically acceptable carrier of for the asymmetric disulfide. Preferably the asymmetric disulfide in the composition is an inhibitor of cellular redox signaling and prevents inhibition of apoptosis. The composition may also include a chemotherapeutic.

The composition may be used in a method of inhibiting cellular growth in conditions such as but not limited to FAP polyps or angiogenisis. The method includes contacting cells in a patient with a therapeutically effective amount of an asymmetric disulfide composition that inhibits cellular growth for at least 3 hours; preferably the composition includes 1-methylpropyl 2-imidazolyl disulfide and a polymer.

Another method of the present invention is treating abnormal cellular activity in a patient by administering to a patient a therapeutically effective amount of a composition that includes an asymmetric disulfide or derivative thereof for three hours or more to reduce patient plasma thioredoxin levels. Preferably the asymmetric disulfide composition includes 1-methylpropyl 2-imidazolyl disulfide in a dose of about 9 mg/m² to about 128 mg/m² and may also include a gelatin or a polymer. To maintain the therapeutically effective concentration of the asymmetric disulfide in the patient, the composition is preferably administered intravenously or orally using a time sustained release composition. The intravenous composition may include additives which slow the absorption of the asymmetric disulfide and permit shorter infusion times. The method may also include a chemotherapeutic or radiological treatment that is administered to the patient. Preferably the chemotherapeutic or radiological treatments are administered such that the asymmetric disulfide composition and the effect of the chemotherapeutic or radiological treatment on the patient are present within the patient at the same time. This method is useful for diseases characterized by over expression of thioredoxin in the patient and may include but is not limited to breast cancer, renal cancer, colon cancer, and glialblastomas.

Another method of the present invention is the prophylactic treatment of a patient comprising for abnormal cellular activity. The method includes administering to a patient a therapeutically effective amount of a composition including an asymmetric disulfide or derivative thereof for three hours or more to prevent abnormal cell activity in the patient. Preferably the asymmetric disulfide in the composition includes 1-methylpropyl 2-imidazolyl disulfide in a dose up to about 250 mg/m² and more preferably 128 mg/m². To maintain the therapeutically effective concentration of the asymmetric disulfide in the patient, the composition is preferably administered intravenously or orally using a time sustained release composition that may include a polymer. The intravenous composition may include additives which slow the absorption of the asymmetric disulfide and permit shorter infusion times.

The examples presented below are intended to illustrate particular embodiments of the invention and are not intended to limit the scope of the specification, including the claims, in any way.

The materials and methods of the present invention are as follows: Enzymes: Thioredoxin reductase, specific activity 43.6 μmole NADPH reduced/mm/mg protein at 21° C., was purified from human placenta as previously described (Oblong et al., 1993). Glutathione reductase, specific activity 141.2 μmole NADPH reduced/mm/mg protein at 21° C., was purified from aged human red blood cells (Colmon & Black, 1965). Human recombinant thioredoxin was expressed in E. coli and purified as previously described (Gasdaska et al., 1994). The thioredoxin was stored at −20° C. with 5 mM dithiothreitol which was removed before use with a desalting column (PD10, Pharmacia, Uppsala, Sweden).

Not only do the asymmetric disulfides studied herein inhibit the growth of cancer cells in vitro, they show in vivo anti-tumor activity against human tumor xenografts in scid mice.

Asymmetrical 2-imidazolyl disulfides which were used as a basis for further development and identified below in Table 1. In Table 1, NADPH oxidation by thioredoxin reductase was measured spectrophotometrically as described with either the disulfide as the electron acceptor (substrate) or with thioredoxin and insulin as the final electron acceptor and the disulfide as inhibitors. The reactions were initiated by the addition of NADPH. Km and Ki values were calculated from Lineweaver-Burk plots of the data.

TABLE 1
Effects of 2-imidazolyl Disulfides
On Human Thioredoxin Activity

| Compound | R | Type | Km(μM) | Ki |
|---|---|---|---|---|
| ethyl 2-imidazolyl | —CH₂CH₃ | substrate | 48.1 | — |
| disulfide (VI-2) | | | | |
| n-butyl 2-imidazolyl | —(CH₂)₃CH₃ | substrate | 43.1 | — |
| disulfide (III-2) | | | | |
| 1-methylpropyl 2- | —CH(CH₃)CH₂CH₃ | inhibitor | — | 30.8 |
| imidazolyl disulfide | | | | |
| (IV-2) | | | | |
| t-butyl 2-imidazolyl | —C(CH₃)₃ | non- | — | — |
| disulfide (IX-2) | | reactive | | |
| benzyl 2-imidazolyl | —CH₂C₆H₅ | inhibitor | — | 30.9 |
| disulfide (DLK-36) | | | | |

The compounds of Table 1 were synthesized by a method described previously and recrystallized prior to use. Stock solutions of the disulfides were prepared at 10 mM in ethanol and diluted in aqueous media just prior to use. N-ethylmaleimide (NEM), diamide, and DTT were obtained from Sigma Chemical Co. (St. Louis, Mo.). All other chemicals used were of reagent grade. Human placental thioredoxin reductase (specific activity 33.3 μmole NADPH reduced/mm/mg at room temperature) was purified as previously described and human glutathione reductase (specific activity 141.2 μmole NADPH reduced/mm/mg at room temperature) was purified from aged human red blood cells.

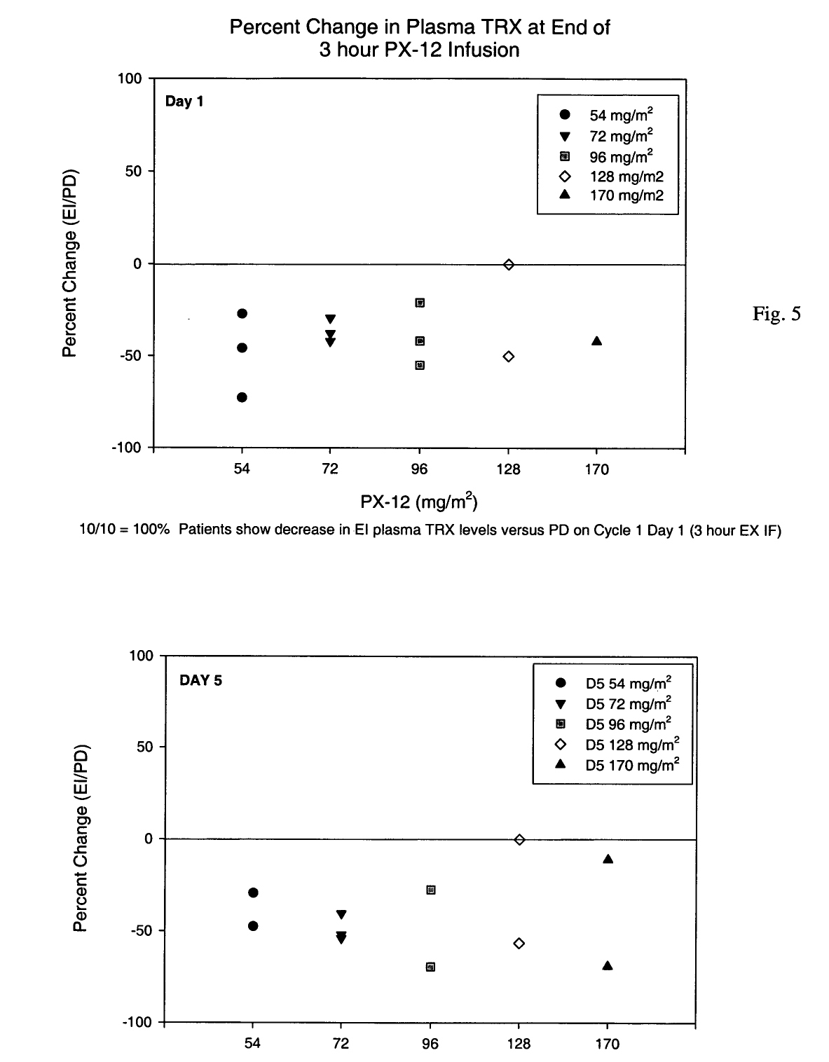

The asymmetric disulfides of the present invention appear to interact with both thioredoxin reductase and thioredoxin. While not intending to be bound by theory, it is postulated that the unbranched alkyl moieties of the disulfides III-2 and VI-2, both of which are substrates for thioredoxin reductase, facilitates thiol/disulfide exchange with a Cys residue at the catalyic site of thioredoxin reductase and the liberation of 2-mercaptoimidazole (FIG. 5A). There is a subsequent thiol/disulfide exchange to give an oxidized catalytic site followed by reduction by NADPH to generate the reduced enzyme. Branching of the alkyl substituent of IV-2 and the benzyl group of DLK 36, prevents these compounds from readily undergoing the second thiol/disulfide exchange so that these agents are weak competitive inhibitors of the TR/Trx system. Extensive branching, as with the t-butyl analog, IX-2, decreased the reactivity of the disulfide towards the catalytic site Cys residues of thioredoxin reductase and prevents IX-2 from acting as either a substrate or inhibitor.

The lead compound, IV-2, and some analogues have been shown to exhibit dose-dependent antitumor activity against human MCF-7 breast cancer and HL-60 xenografts growing in scid mice. This is shown below in Table II.

TABLE 2
Activity against MCF-7 breast cancer cells in scid mice

| | Dosage mg/kg/day | | Toxicity |
|---|---|---|---|
| Compound | for 14 days | T/C^{b} % | dead/injected |
| III-2 | 6 | 48.9^{c} | 0/8 |
| | 12 | 56.6^{c} | 0/8 |
| | 18 | 100.2 | 1/8 |
| IV-2 | 5 | 56.7 | 2/8 |
| | 10 | 45.9^{c} | 0/7 |
| | 15 | 2.3^{c} | 0/7 |
| VI-2 | 4 | 83.0 | 1/8 |
| | 6 | 65.8 | 0/8 |
| | 12 | 64.6 | 4/8 |
| DLK-36 | 25 | 100.7 | 1/8 |
| | 40 | 23.7^{c} | 0/6 |

^{a}i.p. daily for 14 days starting day 1
^{b}tumor volume of treated versus control at day 28
^{c}p < 0.005

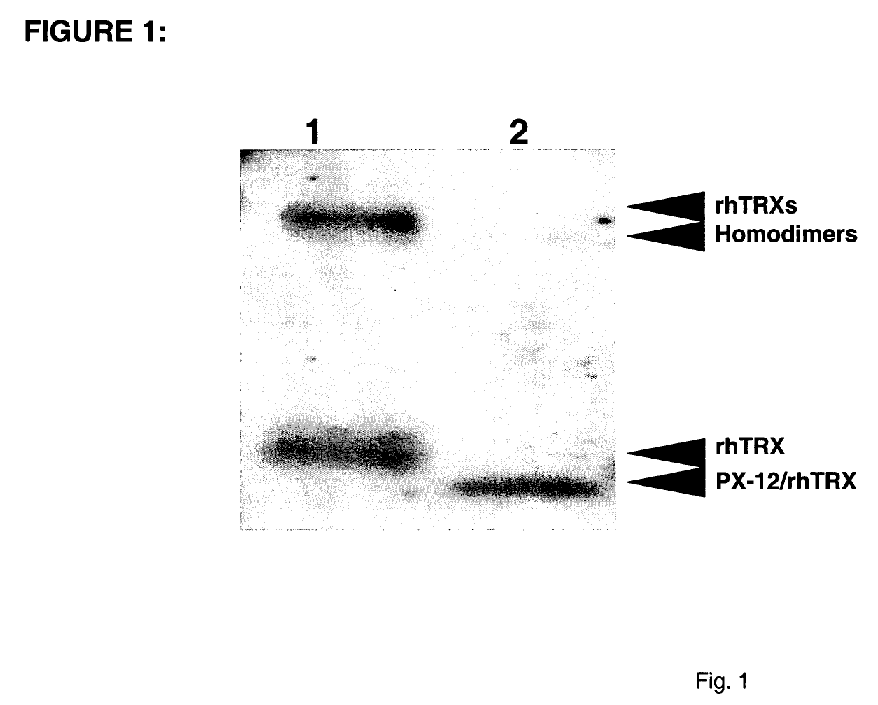

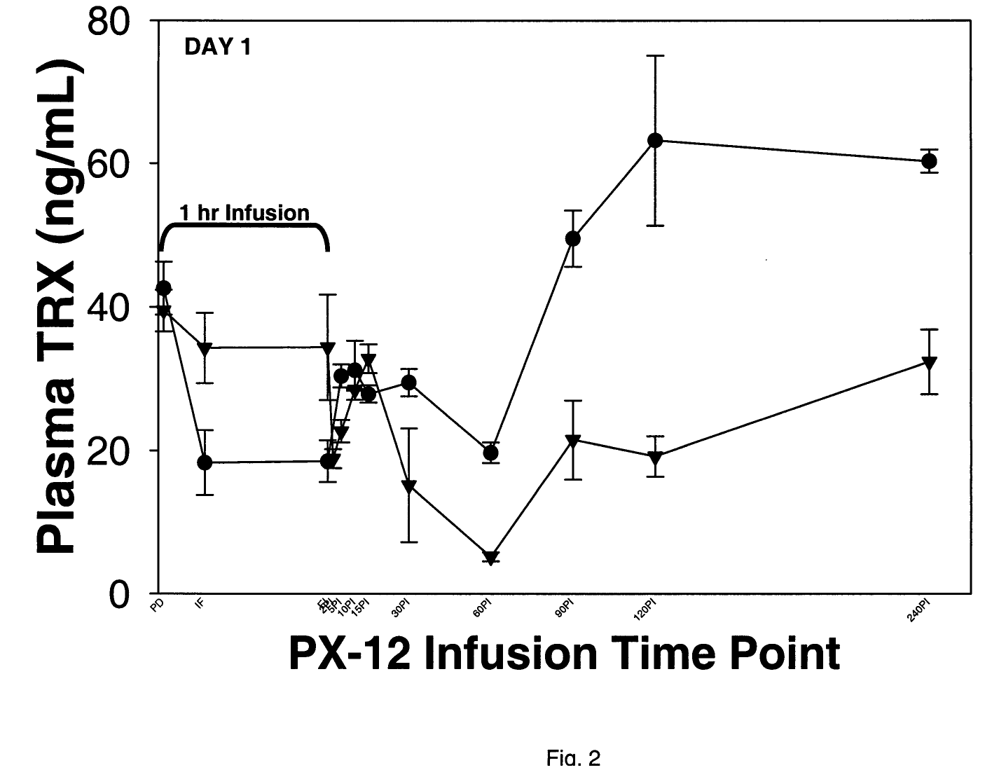

The asymmetric disulfide IV-2, (1-methylpropyl 2-imidazolyl disulfide), which is a thioredoxin (TRX) inhibitor has evaluated in clinical trials (FIG. 1). Its molecular target, TRX, is a ubiquitously expressed redox protein. (11.5 kDa) that is over expressed in various human cancers. Without being bound by theory, IV-2 interacts with the active site of TRX and physically modifies TRX via thioalkylation of conserved cystaine residues within the TRX active site which can alter its electrophoretic mobility (FIG. 2). Western blotting studies show that IV-2 treatment of human carcinoma cell lines induce a dose and time-dependent modification of intracellular TRX. Significantly, immunoprecipitation and Western blotting studies show that after treatment of normal, whole human blood with IV-2, that IV-2 induces a close and time-dependent modification of white blood cells and circulating TRX. Two biological endpoint assays were developed and validated in order to quantitatively measure the effects of IV-2 on plasma TRX.

Presented here are data from the pharmacodynamic (PD) evaluation of the biological activity of IV-2 in plasma from treated patients. Plasma samples were analyzed by TRX ELISA and TRX-specific immunoprecipitation and Western Blot. Both assay methods were used to quantify a IV-2 induced effect on circulating TRX. Patient variability in circulating TRX level was found. Data indicated i.v. infusion of IV-2 induced a time and dose dependent decrease in circulating reduced TRX. ELISA analyses indicated there was a consistent and reproducible reduction in reduced plasma TRX detected. The decrease in reduced TRX levels occurred rapidly and was observed across all IV-2 dose range treatment cohorts. Data comparing 1 hour versus 3 hour IV-2 i.v. infusions showed that the 3-hour PX12 infusion decreased plasma TRX levels more consistently and for longer time periods than the 1 hour IV-2 infusion.

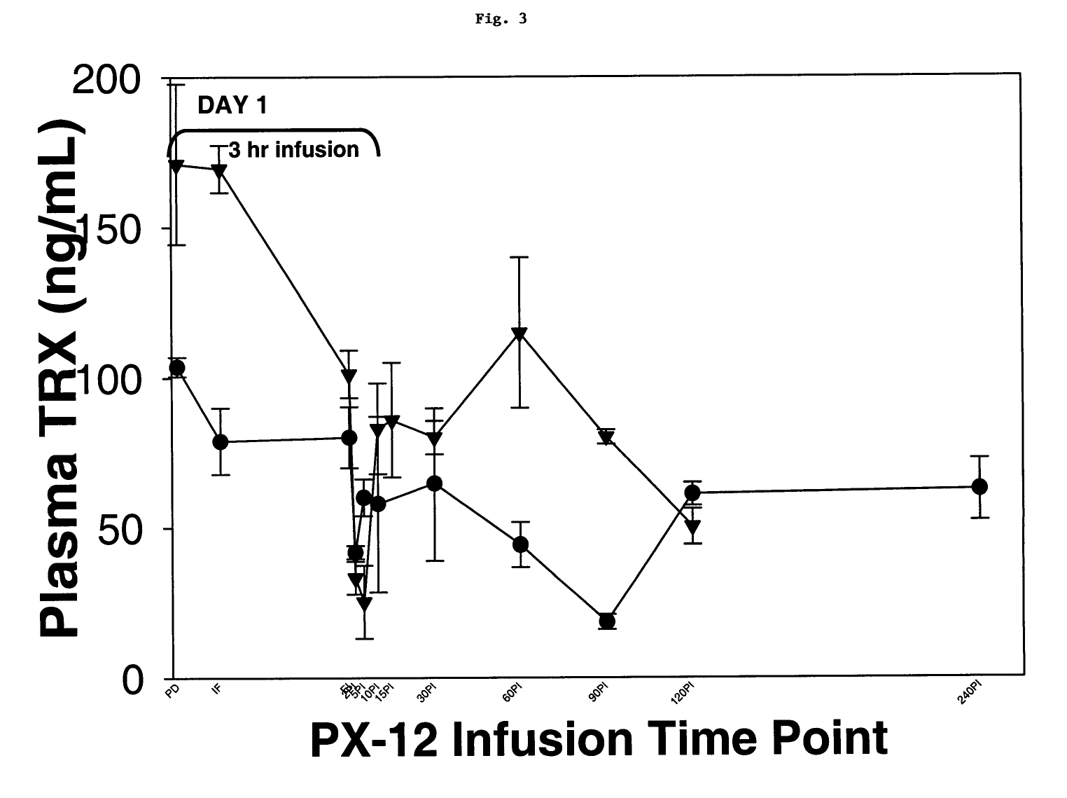

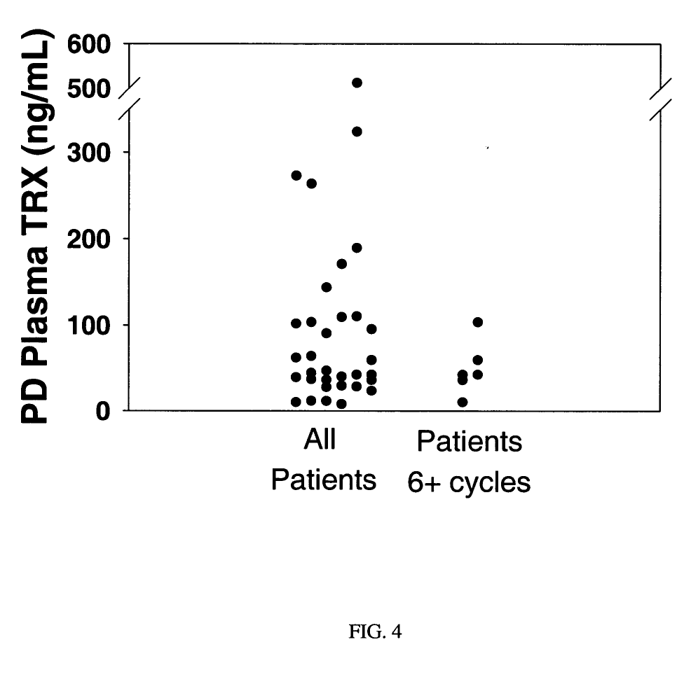

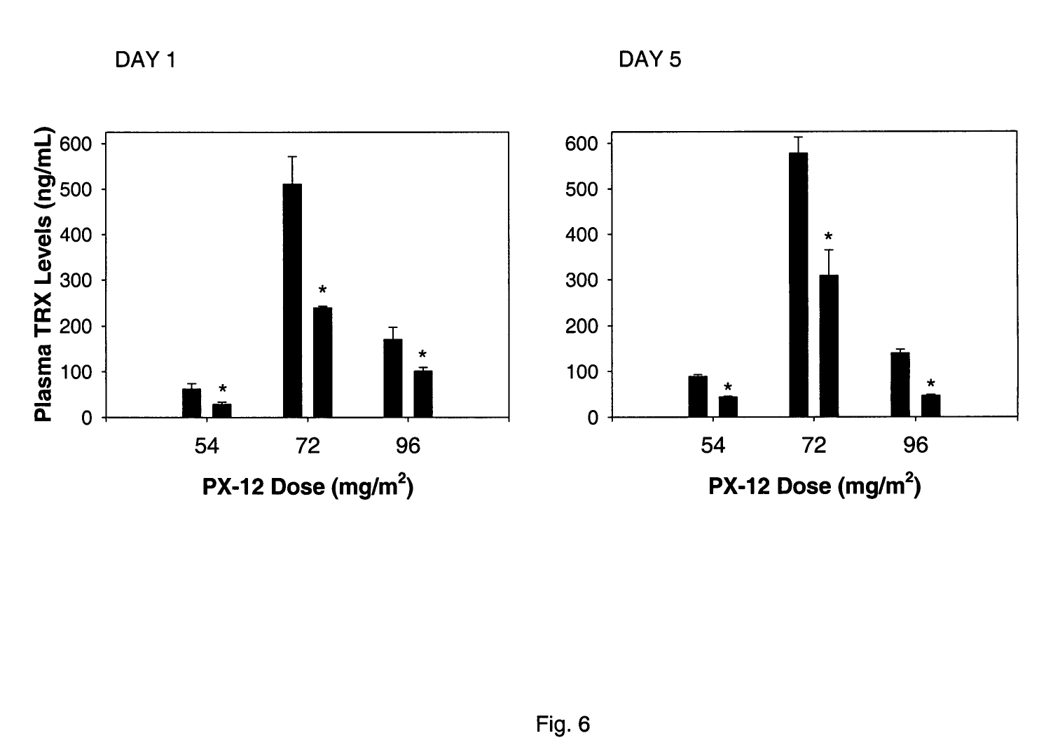

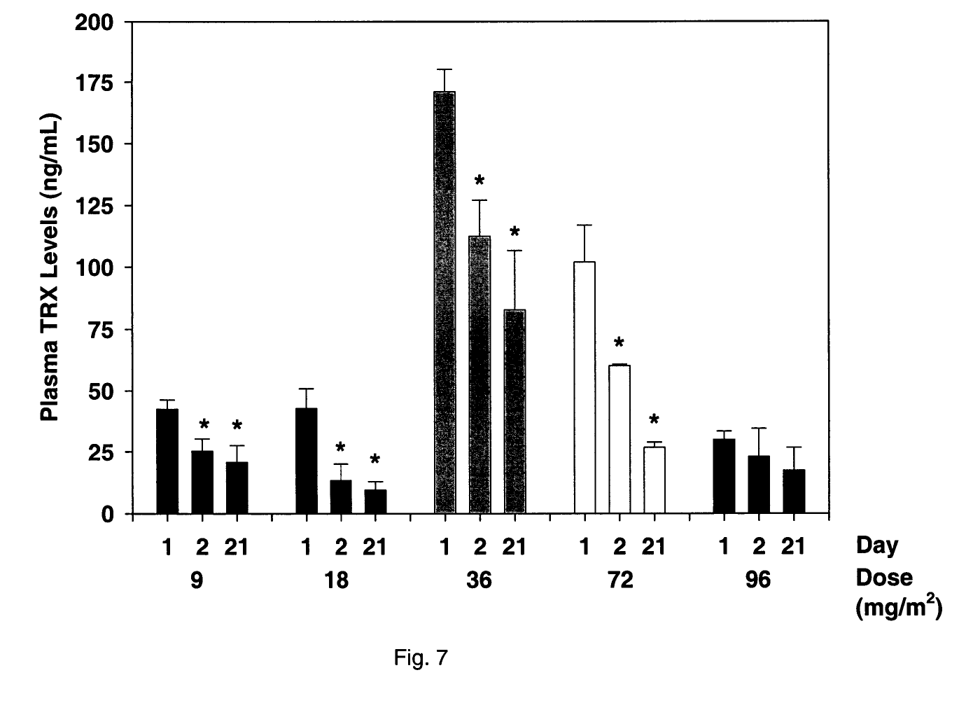

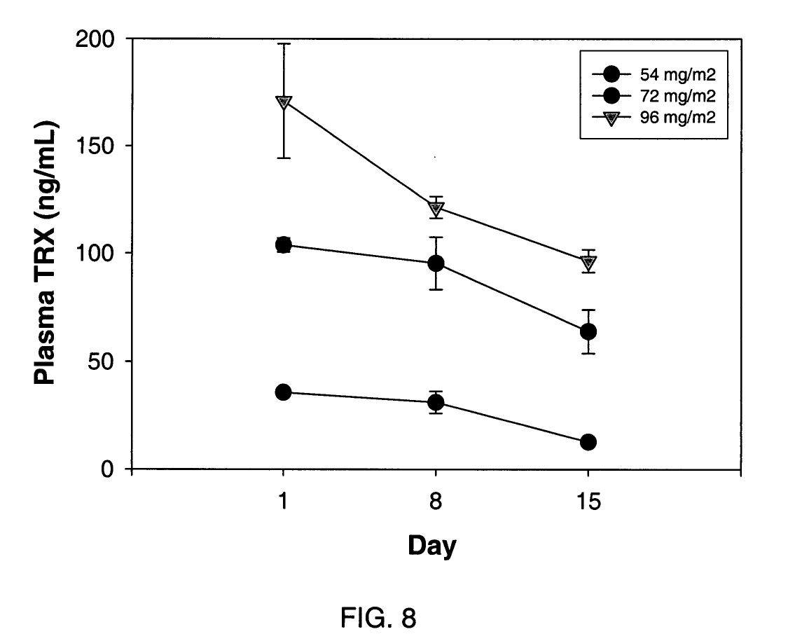

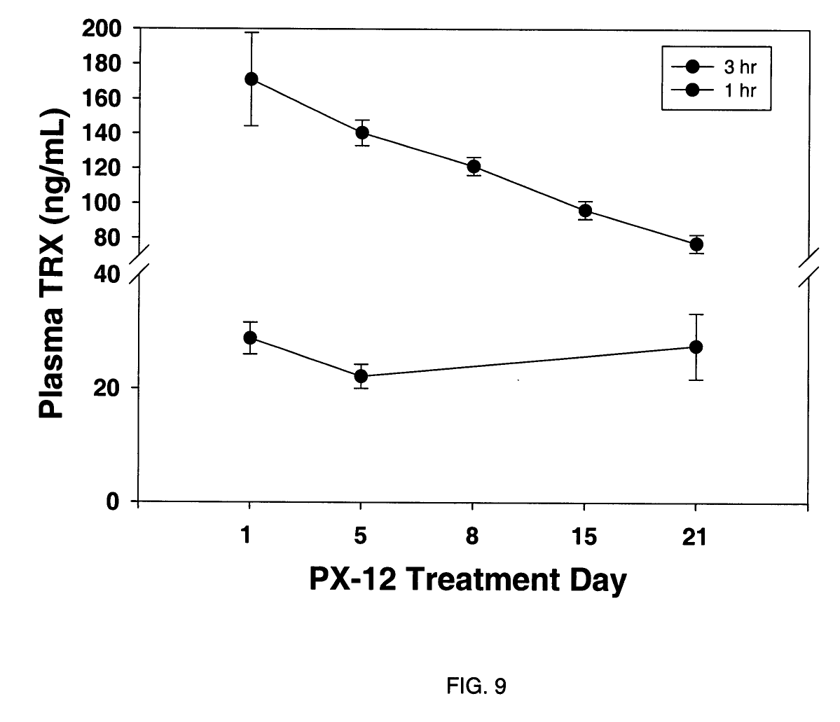

These data provide evidence that IV-2 has a measurable effect on its molecular target, (TRX) in a clinical setting. PD effects on TRX by IV-2 will be compared to patient response. FIG. 1 shows that IV-2 treatment alters rhTRX electrophoretic mobility. Extensive research has shown that IV-2 treatment of rhTRX (lane 1) induces a downshift in rhTRX (lane 2) [UV-2/TRX] electrophoretic gel mobility. This downshift if associated with thioalkylation of conserved cystaine residues within the TRX active site. Subsequent studies demonstrated that IV-2 treatment of human carcinoma cell lines and normal, whole human blood induced the characteristic downshift in intracellular and circulating (plasma) TRX. These and subsequent studies also confirmed that chicken or human TRX recognized IV-2 modified TRX via Western blot and ELISA. FIG. 2 illustrates the plasma thioredoxin was determined, during the infusion, and for 2-4 hours after the end of the infusion. With protocol modification, some of the patients also had their TRX levels evaluated on days 8 and 15 after the infusion. It was found that the plasma TRX was rapidly lowered within the first 15 minutes of the start of the infusion, even at a dose as low as 9 mg/m². The TRX level remained low through the end of the infusion. The levels were found to be pre-dose levels in a dose dependent manner, with the rate of return faster at the lower dosing cohorts as more clearly illustrated in FIG. 2. As illustrated in FIG. 3, when the infusion length was modified to 3 hours, it was found that TRX levels were decreased constantly lower and the length of the decrease was found to be consistently longer than when equilivant doses of PX-12 were delivered over a 1 hour period. FIG. 4 shows a pharmacodynamic anlysis that provides information regarding patients starting TRX levels and how the levels were modified during and after treatment with PX-12. The pre-dosing plasma thioredoxin levels in 25 patients which have been analyzed to date were obtained and compared to those patients who demonstrated stable disease and received six cycles of therapy or more. FIG. 5 shows that 3-hr IV-2 infusion is more effective than 1-hr IV-2 infusion in inducting an immediate decrease in plasma TRX levels. FIG. 6 shows that 3-hr IV-2 infusion results in a significant decrease in plasma TRX at end of infusion as to Pre-Dosing levels across IV-2 treatment cohorts. FIG. 7 illustrates that a 1-hr IV-2 infusion results in a sustained decrease in plasma TRX within cycle 1 and across IV-2 treatment cohorts in some but not all patients. FIGS. 8 and 9 illustrate that the 3 hour delivery of PX-12 also lowered the circulating TRX levels over the entire cycle of therapy.

Current Trial Status is that 28 patients successfully treated in cohorts from 9 to 128 mg/m²; IV-2 was well tolerated with no dose limiting toxicities to date.

The results of the tests illustrate that IV-2 treatment induced a decrease in circulating TRX levels as measured by an ELISA assay; reduction in TRX levels occurred within first 15 minutes of the IV-2 infusion; IV-2 induced decrease in TRX levels was more pronounced and prolonged at higher IV-2 doses; TRX levels were lowered to greater extent and remained decreased over longer period when delivered over longer infusion times; a statistically significant decrease in plasma TRX levels was observed at end of the IV-2 infusion (E1) as compared to TRX levels pre-IV-2 infusion (PD) for all patients treated with a 3-hr infusion.

The unsymmetrical disulfides of the present invention have been shown to inhibit thioredoxin stimulated growth in vitro and display anti-tumor activity in vivo. It has been proposed that the inhibition is the result of thioalkylation of the active site cysteine residues. Disulfide moieties are critical for this inhibition through a thiol/disulfides exchange reaction and the aromatic and alkyl portions of the molecules confer potency and specificity by affecting the rates of reactivity.

It is anticipated that as the understanding of the asymmetric disulfides of the present invention as well as cellular redox systems and their role in the control of cell growth advances, new targets for anti-cancer drug development will emerge. The link between external stimuli and activation of growth, cell death and transformation, through redox modulation is growing. The possibility of reversing the uncontrolled growth of tumors through control of redox signaling, or committing a cell to die by the redox regulation of factors involved in cell death provide intriguing prospects for drug development.

While the foregoing has been set forth in considerable detail, the sequences are presented for elucidation, and not limitation. Modifications and improvements, including equivalents, of the technology disclosed above which are within the purview and abilities of those in the art are included within the scope of the claims appended hereto. It will be readily apparent to those skilled in the art that numerous modifications, alterations and changes can be made with respect to the specifics of the above description without departing from the inventive concept described herein. For example, it is specifically contemplated herein that the asymmetric disulfides may be modified to fluoresce and used as a tag to monitor the thioredoxin/thioredoxin reductase system. Additionally, it is specifically contemplated herein that the disulfides of the present invention may be incorporated into a mesh column to separate or isolate proteins or enzymes of a redox system, particularly a thioredoxin redox system.

What is claimed:

1. An composition for sustained release delivery of an asymmetric disulfide comprising:
an asymmetric disulfide or the derivative thereof;
a matrix including at least one polymer.

2. The composition of claim 1 wherein said composition erodes and releases the asymmetric disulfide or the derivative thereof in a patient and maintains in the patient a therapeutically effective concentration of said asymmetric disulfide in the patient for at least three hours.

3. The composition according to claim 1 wherein said hydrophilic polymer is selected from the group consisting of gelatin, methyl cellulose, hydroxypropyl methylcellulose, hydroxyethyl cellulose, hydroxypropyl cellulose, hydroxyethyl methylcellulose, carboxymethylcellulose and sodium carboxymethylcellulose.

4. The composition of claim 1 further including a pharmaceutically acceptable carrier of said asymmetric disulfide.

5. The composition of claim 1, wherein said asymmetric disulfide is an inhibitor of cellular redox signaling.

6. The composition of claim 5, wherein said inhibitor of cellular redox signaling prevents inhibition of apoptosis.

7. The composition of claim 5, wherein said inhibitor is an inhibitor of thioredoxin or thioredoxin reductase.

8. The composition of claim 1 further comprising a chemotherapeutic.

9. The composition of claim 1, wherein the asymmetric disulfide includes 1-methylpropyl 2-imidazolyl disulfide.

10. A method of inhibiting cellular growth comprised of:
contacting cells in a patient with a therapeutically effective amount of an asymmetric disulfide composition for at least 3 hours, said asymmetric disulfide composition being an inhibitor of cellular growth.

11. The method of claim 10 wherein said asymmetric composition includes 1-methylpropyl 2-imidazolyl disulfide and a polymer.

12. The method of claim 10 wherein the cellular growth is FAP polyps or angiogenisis.

13. A method of treating abnormal cellular activity in a patient, said treatment comprising:
administering to a patient a therapeutically effective amount of a composition including an asymmetric disulfide or derivative thereof for three hours or more to reduce patient plasma thioredoxin levels.

14. The method of claim 13 wherein said asymmetric disulfide includes 1-methylpropyl 2-imidazolyl disulfide in a dose of about 9 to about 128 mg/m².

15. The method of claim 13 wherein said composition includes 1-methylpropyl 2-imidazolyl disulfide and a polymer.

16. The method according to claim 13 wherein said administering step comprises:
intravenous administration of a composition including 1-methylpropyl 2-imidazolyl disulfide to said patient which results in a therapeutically effective amount of the disulfide in the patient for three hours or more.

17. The method according to claim 13 wherein said administering step comprises:
oral administration of a composition including 1-methylpropyl 2-imidazolyl disulfide to said patient which results in a therapeutically effective amount of the disulfide in the patient for three hours or more.

18. The method according to claim 13 wherein a chemotherapeutic or radiological treatment is administered to the patient such that the asymmetric disulfide composition and the effect of the chemotherapeutic or radiological treatment on the patient, are present within the patient at the same time.

19. The method of claim 13 wherein the disease characterized by over expression of thioredoxin in the patient is chosen from the group consisting of breast cancer, renal cancer, colon cancer, and glialblastomas.

20. A prophylactic treatment of a patient comprising:
administering to a patient a therapeutically effective amount of a composition including an asymmetric disulfide or derivative thereof for three hours or more to prevent abnormal cell activity in a patient.

21. The method according to claim 20 wherein said asymmetric disulfide is 1-methylpropyl 2-imidazolyl disulfide in a dose up to about 128 mg/m².

22. The method according to claim 20 wherein said administering step comprises:
intravenous administration of the composition.

23. The method according to claim 20 wherein said administering step comprises:
oral administration of a sustained release composition including 1-methylpropyl 2-imidazolyl disulfide.

24. The method according to claim 20 wherein said composition further includes a polymer.

25. A method of treating pancreatic cancer comprising: administering a therapeutically effective of asymmetric disulfide to achieve a therapeutic purpose.

26. The method of claim 25, wherein said asymmetric disulfide is a imidazolyl disulfide.

27. The method of claim 25, wherein the imidazolyl disulfide is 1-methylpropyl 2-imidazolyl disulfide.
